(19) United States
(12) Patent Application Publication
(10) Pub. No.: US 2025/0205382 A1
(43) Pub. Date: Jun. 26, 2025

(54) PHOTOCATALYTIC AIR PURIFIER AND AIR PURIFICATION METHOD

(71) Applicant: Hamamatsu Pulse Co.,Ltd., Shizuoka (JP)

(72) Inventor: Masahito KONDO, Shizuoka (JP)

(21) Appl. No.: 18/979,023

(22) Filed: Dec. 12, 2024

(30) Foreign Application Priority Data
Dec. 26, 2023 (JP) 2023-218760

(51) Int. Cl. A61L 9/20; B01D 53/00; B01D 53/86; B01D 53/88

(52) U.S. Cl. CPC A61L 9/205; B01D 53/007; B01D 53/8687; B01D 53/885; A61L 2209/12; B01D 2255/20707; B01D 2255/802; B01D 2257/70; B01D 2257/90; B01D 2257/91; B01D 2258/06; B01D 2259/802; B01D 2259/804

(57) ABSTRACT

Provided is a photocatalytic air purifier which includes, in a housing, a photocatalyst that purifies air and a light source that irradiates the photocatalyst with light exerting a photocatalytic action, in which the photocatalyst is elastically supported in the housing. Also provided is an air purification method comprising: using a photocatalytic air purifier including, in a housing, a photocatalyst that purifies air and a light source that irradiates the photocatalyst with light exerting a photocatalytic action; elastically supporting the photocatalyst in the housing; and vibrating the photocatalyst.

### CROSS-REFERENCE TO RELATED APPLICATION

This application is based on Japanese Patent Application No. 2023-218760 filed with the Japan Patent Office on Dec. 26, 2023, the entire content of which is hereby incorporated by reference.

### BACKGROUND

### 1. Technical Field

The present disclosure relates to a photocatalytic air purifier that purifies air using a photocatalyst and an air purification method.

### 2. Related Art

There has been known a photocatalytic air purifier that decomposes an organic substance in air or annihilates a bacterium in air by a photocatalytic action of a photocatalyst, such as titanium oxide, irradiated with light such as ultraviolet light. For example, in a fluid treatment device disclosed as a photocatalytic air purifier in JP-T-2021-511161 (Patent Literature 1), a blower is attached to a frame holding, in a fixed manner, a photocatalytic filter and a light source including a light emitting diode that irradiates a photocatalyst with light.

### SUMMARY

An air purifier according to an embodiment of the present disclosure includes, in a housing, a photocatalyst that purifies air and a light source that irradiates the photocatalyst with light exerting a photocatalytic action, in which the photocatalyst is elastically supported in the housing.

### BRIEF DESCRIPTION OF DRAWINGS

FIG. 1 is a perspective view showing the outline of an external configuration of a photocatalytic air purifier according to the present embodiment;

FIG. 2 is a side view showing the outline of an internal configuration of a purifier body of the air purifier shown in FIG. 1 with a side portion of the purifier body removed for illustration of the internal configuration;

FIG. 3 is a perspective view showing, from a suction port side, the outline of an external configuration of a photocatalytic unit inserted into the purifier body shown in FIG. 2;

FIG. 4 is a perspective view showing, from a discharge port side, the outline of the external configuration of the photocatalytic unit inserted into the purifier body shown in FIG. 2;

FIG. 5 is an exploded perspective view showing a state of first holders of the photocatalytic unit shown in FIG. 3 being detached; and

FIG. 6 is an exploded perspective view for describing the state of arrangement of elastic supports relative to photocatalyst carriers held in the photocatalytic unit shown in FIG. 3.

### DETAILED DESCRIPTION

In the following detailed description, for purpose of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

For the photocatalytic air purifier described in Patent Literature 1 above, improvement in an air purification efficiency of the photocatalytic filter is demanded at all times.

The present embodiment copes with the above-described problems. An object thereof is to provide a photocatalytic air purifier and an air purification method capable of improving an air purification efficiency of a photocatalytic filter.

To achieve the above object, an air purifier according to the present embodiment includes, in a housing, a photocatalyst that purifies air and a light source that irradiates the photocatalyst with light exerting a photocatalytic action, in which the photocatalyst is elastically supported in the housing.

According to the above-described embodiment, in the photocatalytic air purifier, the photocatalyst is elastically supported in the housing. Thus, an opportunity of contact of air with the photocatalyst increases. As a result, an air purification efficiency can be improved.

The photocatalytic air purifier according to the present embodiment may further include an air blower that sends air to a photocatalyst side or sucks air from the photocatalyst side.

According to the above-described embodiment, the photocatalytic air purifier includes the air blower that sends air to the photocatalyst side or sucks air from the photocatalyst side. Thus, the photocatalyst vibrates due to actuation of the air blower itself or an air flow by the air blower. Consequently, the opportunity of contact of air with the photocatalyst increases. As a result, the air purification efficiency can be improved.

Moreover, the photocatalytic air purifier according to the embodiment may further include a photocatalytic unit, in which the photocatalytic unit is attached in the housing, and supports the photocatalyst, and the photocatalyst is elastically supported in the photocatalytic unit.

According to the above-described embodiment, in the photocatalytic air purifier, the photocatalyst is elastically supported in the photocatalytic unit. Thus, the photocatalyst can be attached to the housing through the photocatalytic unit. As a result, manufacturability and maintainability of the photocatalytic air purifier can be improved.

Moreover, in the photocatalytic air purifier according to the embodiment, the photocatalytic unit may be attachable to and detachable from the housing.

According to the above-described embodiment, in the configuration of the photocatalytic air purifier, the photocatalytic unit is attachable to and detachable from the housing. Thus, the manufacturability and maintainability of the photocatalytic air purifier can be improved.

Moreover, in the photocatalytic air purifier according to the present embodiment, the photocatalyst may be directly or indirectly supported, at at least part of an outer peripheral portion of the photocatalyst, by the housing through elastomer or rubber.

According to the above-described embodiment, in the photocatalytic air purifier, at least part of the outer peripheral portion of the photocatalyst is directly or indirectly supported by the housing through the elastomer or the rubber. Thus, the photocatalyst can be easily elastically supported.

The present embodiment can be implemented not only as the embodiment of the photocatalytic air purifier but also as an embodiment of an air purification method.

Specifically, the air purification method includes using a photocatalytic air purifier which includes, in a housing, a photocatalyst that purifies air and a light source that irradiates the photocatalyst with light exerting a photocatalytic action; elastically supporting the photocatalyst in the housing; and vibrating the photocatalyst. According to the air purification method, features and effects similar to those of the disclosure of the photocatalytic air purifier can be expected.

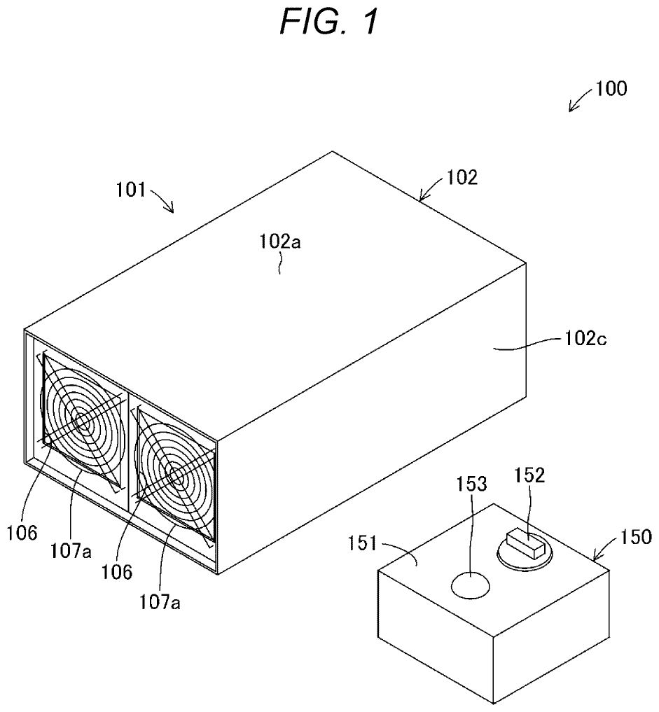

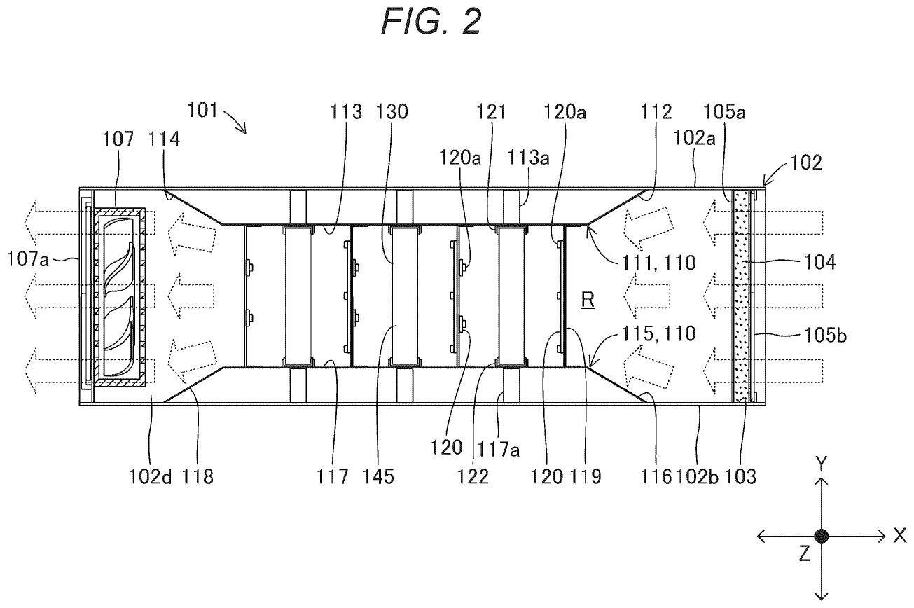

Hereinafter, one embodiment of the photocatalytic air purifier and the air purification method according to the present embodiment will be described with reference to the drawings. FIG. 1 is a perspective view showing the outline of an external configuration of a photocatalytic air purifier (hereinafter merely referred to as an “air purifier”) 100 according to the present embodiment. Moreover, FIG. 2 is a side view showing the outline of an internal configuration of the air purifier 100 shown in FIG. 1. For illustration of the internal configuration, a side portion 102c is removed.

### (Configuration of Air Purifier 100)

The air purifier 100 decomposes an organic substance (including an odor component) contained in air by a photocatalytic action. Alternatively, the air purifier 100 annihilates a bacterium (including a virus) contained in air by the photocatalytic action. The air purifier 100 is a device that removes, deodorizes, or eliminates an allergenic substance in this manner. The air purifier 100 mainly includes a purifier body 101 and an operation box 150.

The purifier body 101 is a device that purifies sucked air and then discharges the purified air. The purifier body 101 includes a housing 102.

The housing 102 is a component that mainly houses an air blower 107, a light source board 120, and a photocatalytic unit 130. The housing 102 is configured such that a plate material made of metal (e.g., aluminum) or resin (e.g., polypropylene) is formed in a box shape having a cavity inside. In the present embodiment, the housing 102 is configured such that a metal plate is formed in a rectangular parallelepiped shape. In this case, the housing 102 is configured such that four side portions 102a, 102b, 102c, 102d extending parallel with a longitudinal direction are individually detachable. The housing 102 is formed with a suction port 103 in one of both end surfaces in the longitudinal direction. Moreover, the housing 102 is formed with a discharge port 106 in the other end surface.

Here, the longitudinal direction of the housing 102 is defined as an X-axis direction. A width direction perpendicular to the X-axis direction is defined as a Y-axis direction. An up-down direction perpendicular to the X-axis direction and the Y-axis direction as shown in the figure is defined as a Z-axis direction.

The suction port 103 is an opening. External air which is air outside the housing 102 is introduced into the housing 102 through this opening. In the present embodiment, the suction port 103 is formed such that the substantially entirety of one of both end surfaces of the rectangular parallelepiped housing 102 in the longitudinal direction thereof is opened. A filter 104 is provided in the suction port 103 through a filter support plate 105a and a pressing member 105b.

The filter 104 is a component that filters dirt or dust contained in air introduced through the suction port 103. The filter 104 is formed of a porous body made of resin (e.g., polyurethane). In the present embodiment, the filter 104 has such a size that the suction port 103 is covered with the filter 104, and is formed in a sheet shape having a rectangular shape in front view.

The filter support plate 105a is a component that supports the sheet-shaped filter 104. The filter support plate 105a is formed of a metal flat plate body having a plurality of through-holes. Further, the filter support plate 105a is attached to an edge portion of the suction port 103 of the housing 102. The pressing member 105b is a component that elastically presses the filter 104 against the filter support plate 105a. The pressing member 105b includes two metal wire rods crossing each other and bent in a V-shape. The pressing member 105b is attachably and detachably attached to an edge portion of the suction port 103.

The discharge port 106 is an opening. Air introduced into the housing 102 through the suction port 103 is discharged to the outside of the housing 102 through this opening. In the present embodiment, the discharge port 106 includes two quadrangular openings in the other one (end surface opposite to that for the suction port 103) of both end surfaces of the rectangular parallelepiped housing 102 in the longitudinal direction thereof. The air blower 107 is provided in each of these discharge ports 106.

The air blower 107 is a device that sucks external air into the housing 102 through the suction port 103. In addition, the air blower 107 sends air sucked into the housing 102 to the outside of the housing 102 through a photocatalyst. In the present embodiment, the air blower 107 includes two electric fans. Further, the air blower 107 is attached to the inner surface of each of the two discharge ports 106. A mesh-shaped guard 107a that suppresses a foreign substance from entering the air blower 107 is provided on the outer surface of the air blower 107. An air path forming member 110 is formed inside the housing 102.

The air path forming member 110 is a component that forms a flow path R for guiding air taken into the housing 102 through the suction port 103 to the discharge port 106. In addition, the air path forming member 110 holds the light source board 120 and the photocatalytic unit 130. The air path forming member 110 is configured such that a plate material made of metal (e.g., aluminum) or resin (e.g., polypropylene) is incorporated in an orifice shape. More specifically, the air path forming member 110 includes an upper half body 111, a lower half body 115, and a coupling body 119. In this case, the upper half body 111 and the lower half body 115 are formed vertically symmetrically. Thus, the upper half body 111 will be mainly described. The lower half body 115 will be supplementally described as necessary.

The upper half body 111 is formed in such a manner that a metal (e.g., aluminum) plate material is bent at both end portions. Further, the upper half body 111 is formed so as to extend over the substantially entirety of the housing 102 in the width direction (Y-axis direction) perpendicular to the longitudinal direction of the housing 102. The upper half body 111 mainly includes a windward plate 112, a holding base 113, and a leeward plate 114.

The windward plate 112 is a plate-shaped portion that guides air introduced through the suction port 103 to a center portion of the housing 102. The windward plate 112 has an inclined surface extending downward to the center from the upper inner wall surface of the housing 102 as shown in the figure. Thus, as in the windward plate 112, a windward plate 116 of the lower half body 115 also has an inclined surface extending upward to the center from the lower inner wall surface of the housing 102 as shown in the figure. Thus, the flow path R is formed so as to be narrowed (decreased in a sectional area) from the suction port 103 side to the holding base 113, 117 side.

The holding base 113 is a portion that holds, together with the holding base 117 of the lower half body 115, both end portions of the photocatalytic unit 130. The holding base 113 is formed in a plate shape parallel with the X-Y plane as shown in the figure. In this case, the holding base 113 is supported through a cylindrical support boss 113a at a position apart downward from the inner wall surface (upper inner wall surface in the figure) of the housing 102 facing the holding base 113 as shown in the figure.

Thus, the holding base 117 of the lower half body 115 is similarly supported through a cylindrical support boss 117a at a position apart upward from the inner wall surface (lower inner wall surface in the figure) of the housing 102 facing the holding base 117 as shown in the figure. With this configuration, the flow path R extends to the leeward plate 114 side in a state of the sectional area narrowed by the windward plates 112, 116 being maintained constant. An interval in the Z-axis direction between the holding base 113 and the holding base 117 as shown in the figure is set to such an interval that the holding base 113 and the holding base 117 can sandwich and hold the photocatalytic unit 130. The holding base 113 is provided with a unit holder 121. Similarly, the holding base 117 is provided with a unit holder 122. The holding base 113 and the holding base 117 are coupled to each other through the coupling body 119.

The leeward plate 114 is a plate-shaped portion that guides air having flowed between the holding base 113 and the holding base 117 to the air blower 107. The leeward plate 114 has an inclined surface extending upward from the center of the housing 102 as shown in the figure. Thus, as in the leeward plate 114, a leeward plate 118 of the lower half body 115 also has an inclined surface extending downward from the center of the housing 102 as shown in the figure. With this configuration, the flow path R is formed so as to be widened (increased in a sectional area) from the holding base 113, 117 side to the air blower 107 side.

The coupling body 119 is a component that couples the upper half body 111 and the lower half body 115 to each other through the holding bases 113, 117. In addition, the coupling body 119 holds the light source board 120. The coupling body 119 is formed of a metal (e.g., aluminum) or resin (e.g., polypropylene) plate material. More specifically, the section of the coupling body 119 is in a backwards C-shape formed by bending both end portions of the plate material. The coupling body 119 is coupled to the inner surfaces of the holding base 113 and the holding base 117 facing each other.

In this case, the coupling body 119 is formed with a plurality of openings (not shown) for ensuring an air flow in the flow path R. Moreover, the coupling body 119 is formed in a grid shape in front view. Further, in this example, a plurality (four in the present embodiment) of the coupling bodies 119 is provided at equal intervals in the longitudinal direction of the housing 102 in a space between the holding base 113 and the holding base 117. The light source board 120 is attached to each of these coupling bodies 119.

The light source board 120 is a device that emits light (ultraviolet light or visible light) for exerting the photocatalytic action to the photocatalyst. The light source board 120 includes a plurality of light sources 120a on a printed circuit board. In the present embodiment, the light source board 120 includes a plurality of light sources 120a which is LEDs that emit blue visible light. The light source board 120 is attached to a grid portion provided on the surface of the coupling body 119 facing the photocatalytic unit 130. Here, the grid portion is adjacent to the opening in the surface facing the photocatalytic unit 130. That is, the light source boards 120 are each provided on the windward and leeward sides of the flow path R with one photocatalytic unit 130 interposed therebetween.

The unit holders 121, 122 are components that hold the photocatalytic unit 130. The unit holder 121, 122 is formed of a metal (e.g., aluminum) or resin (e.g., polypropylene) plate material, in a groove shape with a backwards C-shaped (U-shaped) section. The unit holder 121 is attached to the surface of the holding base 113 facing the holding base 117. Similarly, the unit holder 122 is attached to the surface of the holding base 117 facing the holding base 113. As described above, the unit holders 121, 122 attached to the holding bases 113, 117 face each other, and extend in the width direction (Y-axis direction as shown in the figure) of the housing 102.

That is, the pair of unit holders 121, 122 facing each other holds one photocatalytic unit 130. In this case, the photocatalytic unit 130 slides in the grooves of the unit holders 121, 122. Thus, the unit holders 121, 122 are formed such that the photocatalytic unit 130 is easily slidable therein. In the present embodiment, three pairs of unit holders 121, 122 are provided in the longitudinal direction of the housing 102.

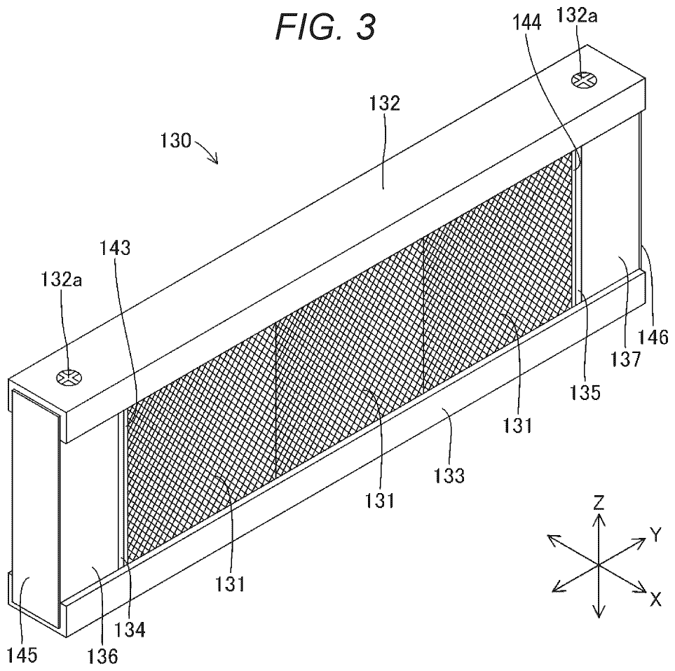

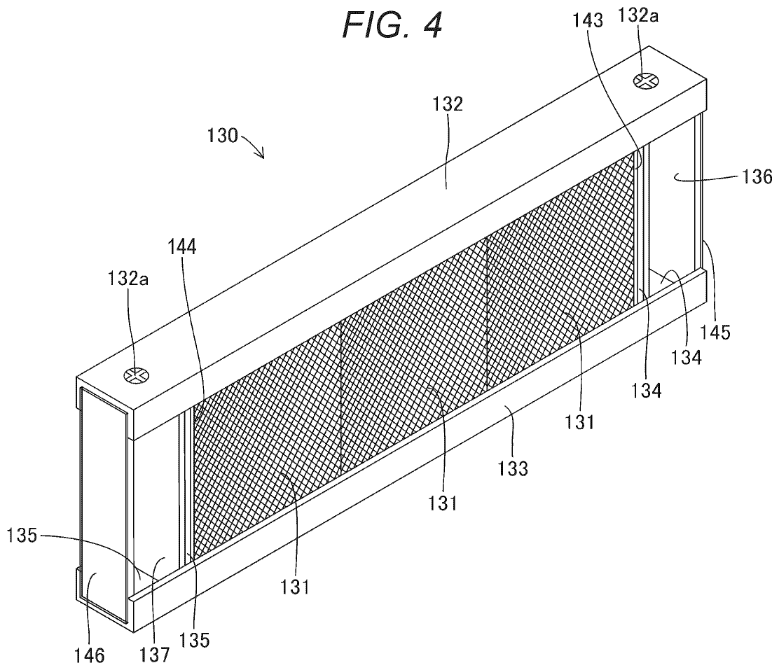

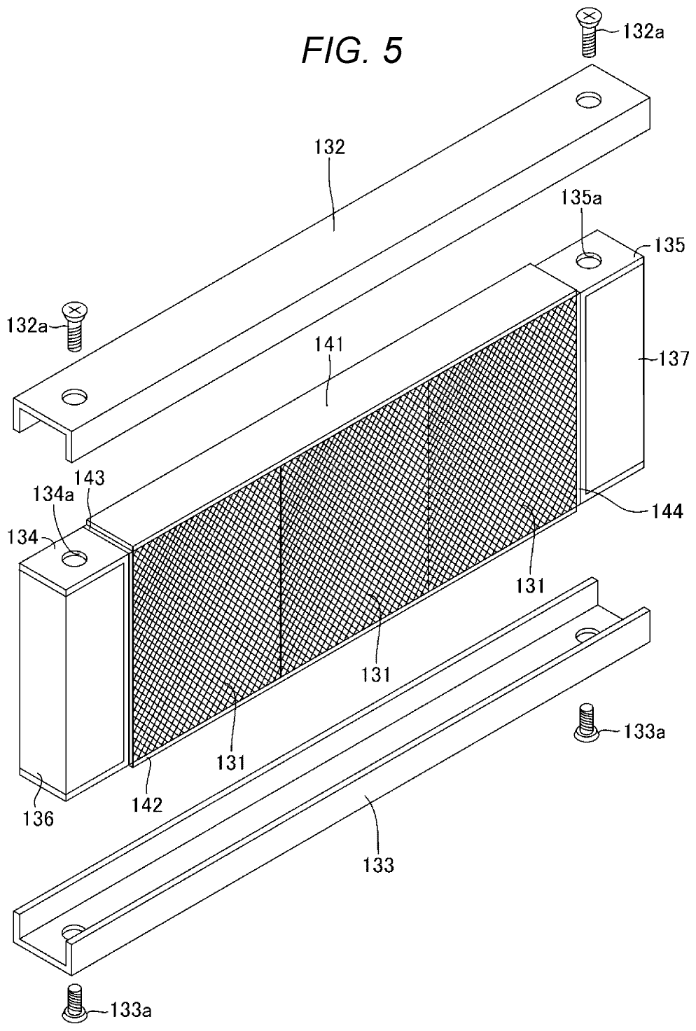

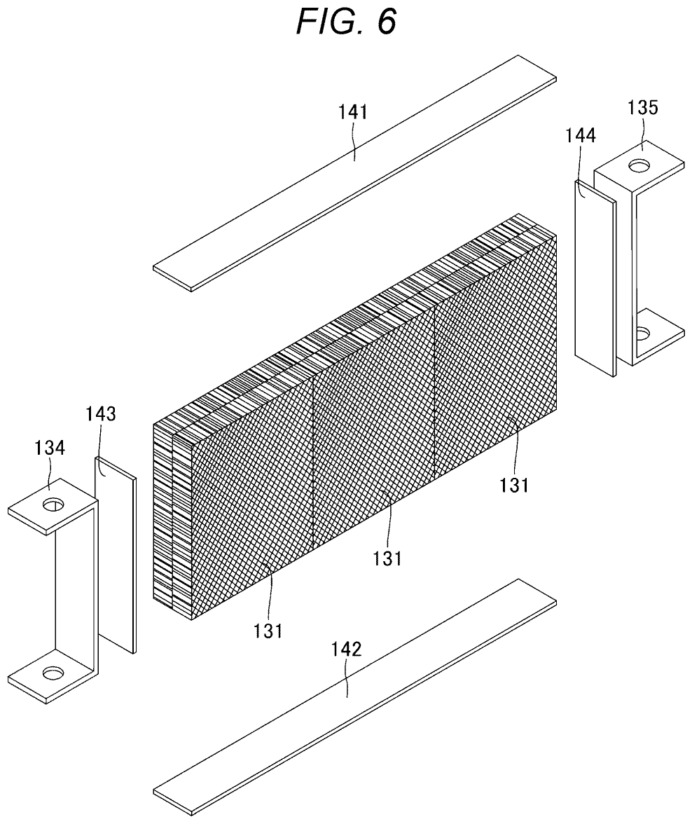

As shown in FIGS. 3 to 6, the photocatalytic unit 130 is a component that holds a photocatalyst carrier 131 and is attachably and detachably fitted in the housing 102. The photocatalytic unit 130 mainly includes the photocatalyst carrier 131, first holders 132, 133, second holders 134, 135, blocking members 136, 137, and elastic supports 141, 142, 143, 144, 145, 146.

The photocatalyst carrier 131 is a component that carries the photocatalyst. Specifically, the photocatalyst carrier 131 is configured such that titanium oxide for exerting the photocatalytic action is applied to a ceramic material (e.g., alumina), a resin material (e.g., silicone resin), a metal material (e.g., aluminum material), or an activated carbon sheet. In the present embodiment, the photocatalyst carrier 131 is configured such that titanium oxide is applied to an activated carbon corrugated thin plate-shaped honeycomb plate having a quadrangular shape in plan view. That is, the photocatalyst carrier 131 is configured such that air flows in a plate surface direction. The photocatalyst carrier 131 is equivalent to the photocatalyst according to the present embodiment.

The first holders 132, 133 are components that sandwich and hold the photocatalyst carrier 131. The first holder 132, 133 is formed in a groove shape with a backwards C-shaped (U-shaped) section from a metal (e.g., aluminum) or resin (e.g., polypropylene) plate material. In the present embodiment, the first holder 132, 133 is formed with such a groove width that two photocatalyst carriers 131 stacked on each other are insertable thereinto. Moreover, the first holder 132, 133 is formed with such a length that three photocatalyst carriers 131 and two second holders 134, 135 arranged on the same plane are insertable thereinto. Each end portion of the first holder 132 is formed with a through-hole into which a screw 132a is inserted. Similarly, each end portion of the first holder 133 is formed with a through-hole into which a screw 133a is inserted.

The second holders 134, 135 are components that sandwich and hold the photocatalyst carrier 131 from a direction perpendicular to the first holders 132, 133. The second holder 134, 135 is formed of a metal (e.g., aluminum) or resin (e.g., polypropylene) plate material bent so as to have a backwards C-shaped (U-shaped) section. In the present embodiment, the second holder 134, 135 is formed with the substantially same width as the thickness of the two photocatalyst carriers 131 stacked on each other. Moreover, the second holder 134, 135 is formed with the substantially same length as that of one side of the photocatalyst carrier 131. Each bent portion of the second holder 134 is formed with an internal thread hole 134a in which the screw 132a is fitted. Similarly, each bent portion of the second holder 135 is formed with an internal thread hole 135a in which the screw 133a is fitted.

The blocking member 136, 137 is a component that suppresses an air flow in an internal space of the second holder 134, 135. The blocking member 136, 137 is formed of a metal (e.g., aluminum) or resin (e.g., polypropylene) plate material bent in a groove shape with a backwards C-shaped (U-shaped) section. In the present embodiment, the blocking member 136, 137 is formed with such width and length that the blocking member 136, 137 is fitted in the internal space of the second holder 134, 135.

The elastic supports 141, 142, 143, 144 are components that elastically support the photocatalyst carrier 131 in the photocatalytic unit 130. The elastic support 141, 142, 143, 144 is made of an elastomer material (e.g., urethane foam resin) or a rubber material. In the present embodiment, the elastic supports 141 to 144 have elasticity, and is formed in a sheet shape from EPDM sponge (ethylene-propylene rubber sponge).

The elastic support 141 of these elastic supports 141 to 144 is bonded, with a double-sided tape or an adhesive, to one of two sides, which are sandwiched between the first holders 132, 133, of four sides of the photocatalyst carrier 131. Similarly, the elastic support 142 is bonded, with a double-sided tape or an adhesive, to the other one of the two sides sandwiched between the first holders 132, 133. The elastic support 143 is bonded, with a double-sided tape or an adhesive, to the second holder 134 of the second holders 134, 135 sandwiching the remaining two sides of the four sides of the photocatalyst carrier 131. Similarly, the elastic support 144 is bonded to the second holder 135 with a double-sided tape or an adhesive. That is, the elastic supports 141 to 144 are arranged between the photocatalyst carrier 131 and the first holders 132, 133 and between the photocatalyst carrier 131 and the second holders 134, 135.

The elastic supports 145, 146 are components that elastically support the photocatalytic unit 130 in the unit holders 121, 122. The elastic support 145, 146 is made of an elastomer material (e.g., urethane foam resin) or a rubber material. In the present embodiment, as in the elastic supports 141 to 144, the elastic support 145, 146 is formed of EPDM sponge (ethylene-propylene rubber sponge) in a sheet shape.

The elastic support 145 is bonded to the outer surface of the blocking member 136 with a double-sided tape or an adhesive. Similarly, the elastic support 146 is bonded to the outer surface of the blocking member 137 with a double-sided tape or an adhesive. That is, the elastic supports 145, 146 are arranged between the photocatalytic unit 130 and the side portions 102c, 102d. This also reduces excessive rattling of the photocatalytic unit 130 in the unit holders 121, 122.

The photocatalytic unit 130 includes the pair of photocatalyst carriers 131 stacked on each other. Specifically, the elastic supports 141, 142 are bonded to three pairs of photocatalyst carriers 131 arranged on the same plane. That is, the photocatalytic unit 130 is sandwiched between the first holder 132 and the first holder 133. In addition, the photocatalytic unit 130 is sandwiched between the second holder 134 and the second holder 135. In this case, the elastic support 143 is bonded to the second holder 134. Similarly, the elastic support 144 is bonded to the second holder 135. In addition, the blocking member 136 is fitted in the internal space of the second holder 134. Similarly, the blocking member 137 is fitted in the internal space of the second holder 135.

The first holder 132, 133 and the second holder 134, 135 are coupled to each other with the screw 132a, 133a. That is, in the present embodiment, the outer periphery of the six photocatalyst carriers 131 arranged in 2× 3 is sandwiched by the first holders 132, 133 and the second holders 134, 135 through the elastic supports 141 to 144. In this state, the photocatalyst carriers 131 are held in the photocatalytic unit 130. Thus, these components of the photocatalytic unit 130 are integrated.

Note that normally, the two photocatalyst carriers 131 are stacked on each other such that the through-holes through which air passes are alternately arranged without overlapping with each other. However, the two photocatalyst carriers 131 can also be stacked on each other such that the through-holes through which air passes are arranged so as to overlap with each other.

The operation box 150 is equipment that controls actuation of the purifier body 101. Specifically, the operation box 150 includes a housing 151 having an operator 152 and an actuation lamp 153. The housing 151 is formed of a metal material (e.g., aluminum material) or a resin material (e.g., polypropylene material) in a box shape.

The operator 152 is a switch component that starts or stops actuation of the air blower 107 and the light source 120a by a user of the air purifier 100. The actuation lamp 153 is a display device to be turned on or off to notify the user of actuation states of the air blower 107 and the light source 120a. The operation box 150 has a power source. The power source is supplied with power from an external power supply source (e.g., a household 100V power source) through a power cord (not shown). The power source supplies the power to the air blower 107 and the light source 120a. The operation box 150 is electrically connected to the purifier body 101 through a not-shown cable.

Note that the purifier body 101 and the operation box 150 may be connected to each other not in a wired manner but in a wireless manner. In this case, the purifier body 101 may have a power source independent of the operation box 150. With this configuration, the purifier body 101 can receive the power from the external power supply source. In the air purifier 100, the purifier body 101 and the operation box 150 may be integrated.

### (Actuation of Air Purifier 100)

Next, actuation of the air purifier 100 configured as described above will be described. The user of the air purifier 100 places the air purifier 100 at a location targeted for air purification. The air purifier 100 may be placed in a building or a closed space such as a house, an office, a public facility, a school, a hospital, a vehicle, a restaurant, a commercial facility, a breeding facility for animals and plants, an agricultural house, or a food storage. In this case, the purifier body 101 can be directly or indirectly attached to a floor surface, a wall surface, or a ceiling surface. In addition, the purifier body 101 can also be disposed in an unfixed manner on a floor, a desk, or a shelf. Note that the air purifier 100 is preferably placed indoor. However, the air purifier 100 can also be placed outdoor.

The user operates the operator 152 of the operation box 150, thereby starting actuating the purifier body 101. When the operator 152 is operated, the purifier body 101 starts actuating the air blower 107 and the light source 120a. Accordingly, in the purifier body 101, the air blower 107 introduces external air into the housing 102 through the suction port 103. In addition, the light source 120a emits visible light to the photocatalyst carrier 131.

The air introduced into the housing 102 through the suction port 103 flows toward the discharge port 106 along the flow path R formed inside the air path forming member 110 (see dashed arrows in FIG. 2). Specifically, while the air in the flow path R being increased in a flow velocity by the windward plates 112, 116, the air is guided to a region between the holding base 113 and the holding base 117 and passes through the photocatalyst carrier 131. In this case, the photocatalyst carrier 131 is irradiated with the visible light for exerting the photocatalytic action from the light source 120a of the light source board 120.

Accordingly, the photocatalyst carrier 131 purifies the air passing through the photocatalyst carrier 131 by the photocatalytic action. In this case, the photocatalyst carrier 131 is elastically supported by the elastic supports 141 to 144 in the photocatalytic unit 130. Thus, in the photocatalytic unit 130, the photocatalyst carrier 131 vibrates due to vibration caused by actuation of the air blower 107 or an air flow brown to the photocatalyst carrier 131. Accordingly, an opportunity of contact between the photocatalyst carrier 131 and the air passing through the photocatalyst carrier 131 increases. This improves a purification efficiency.

The air purified by passing through the three photocatalytic units 130 is discharged to the outside of the housing 102 through the air blower 107 and the discharge port 106, while decreasing in a flow velocity by the leeward plates 114, 118. Accordingly, the air in the space where the air purifier 100 is placed is purified.

At the end of use of the air purifier 100, the user operates the operator 152 of the operation box 150. Accordingly, actuation of the purifier body 101 can be stopped. When the operator 152 is operated, the purifier body 101 stops actuating the air blower 107 and the light source 120a. Accordingly, the user can end air purification by the air purifier 100.

The user can replace the photocatalytic unit 130. Specifically, the user may detach the side portion 102c and/or side portion 102d of the housing 102 to slidably displace the photocatalytic unit 130 along the unit holders 121, 122. In this manner, the user can take out the photocatalytic unit 130. Thus, the user can replace the photocatalytic unit 130 by inserting a new photocatalytic unit 130 into the unit holders 121, 122. Then, the user attaches the side portion 102c and/or the side portion 102d to the housing 102 again. In this manner, the user can end a process of replacing the photocatalytic unit 130.

Note that the user may attach the side portion 102c and/or the side portion 102d to the housing 102 again without inserting the new photocatalytic unit 130 into the unit holders 121, 122 after the photocatalytic unit 130 has been taken out. In this manner, the user can also end the process of replacing the photocatalytic unit 130. Thus, the user can lower an air purification capacity of the air purifier 100. That is, the user can adjust the air purification capacity of the air purifier 100 according to the number of photocatalytic units 130 to be set to the three pairs of unit holders 121, 122.

As can be understood from the description of the actuation above, according to the above-described embodiment, in the air purifier 100, the photocatalyst carrier 131 including the photocatalyst is elastically supported in the housing 102. Thus, the opportunity of contact of air with the photocatalyst increases. Consequently, the air purification efficiency can be improved.

Further, the present embodiment to be implemented is not limited to one described above. Various changes can be made to the above-described embodiment without departing from the object of the present embodiment.

For example, in the above-described embodiment, in the configuration of the air purifier 100, the three photocatalytic units 130 are provided. However, the number of photocatalytic units 130 in the air purifier 100 may be set as necessary according to specifications for air purification. The air purifier 100 may be configured to include at least one photocatalytic unit 130. Thus, in the configuration of the air purifier 100, one pair of unit holders 121, 122 may be provided for one photocatalytic unit 130. In another configuration, plural pairs of unit holders 121, 122 may be provided for one or more photocatalytic units 130.

In the above-described embodiment, in the configuration of the photocatalytic unit 130, the six photocatalyst carriers 131 are provided. However, the configuration of the photocatalytic unit 130 is only required to include at least one photocatalyst carrier 131.

In the above-described embodiment, in the configuration of the air purifier 100, the photocatalyst carrier 131 is provided in the housing 102 through the photocatalytic unit 130. However, in the air purifier 100, the photocatalyst carrier 131 may be directly attached to the housing 102 in a fixed or attachable and detachable manner without the photocatalytic unit 130. In this case, the photocatalyst carrier 131 is attached to the housing 102 through the elastic supports 141 to 144.

In the above-described embodiment, in the configuration of the air purifier 100, the photocatalytic unit 130 is attachable to and detachable from the housing 102. However, in the configuration of the air purifier 100, the photocatalytic unit 130 may be fixed to the housing 102 in an undetachable manner.

In the above-described embodiment, in the air purifier 100, the photocatalyst carrier 131 is disposed in the orientation perpendicular to the direction of the air flow in the housing 102. However, in the air purifier 100, the photocatalyst carrier 131 can also be disposed in an orientation crossing the direction of the air flow in the housing 102 at an angle other than a right angle or an orientation parallel with the direction of the air flow.

In the above-described embodiment, in the configuration of the air purifier 100, the air blower 107 is provided in the housing 102. However, in the configuration of the air purifier 100, the air blower 107 provided outside the housing 102 may send air into the housing 102. In the configuration of the air purifier 100, the air blower 107 may be omitted. In this case, air contacts the photocatalyst carrier 131 by a natural air flow.

In the above-described embodiment, in the configuration of the elastic supports 141 and 142, the elastic supports 141 and 142 are arranged such that the two photocatalyst carriers 131 stacked on each other share these elastic supports 141 and 142. However, in the configuration of the elastic supports 141 and 142, independent elastic supports 141 and 142 may be separately arranged for each two photocatalyst carriers 131 stacked on each other.

In the above-described embodiment, in the configuration of the elastic supports 141 to 144, the elastic supports 141, 142 are bonded to the photocatalyst carrier 131. In addition, the elastic supports 143, 144 are bonded to the second holders 134, 135. However, in the configuration of the elastic supports 141 to 144, the elastic supports 141, 142 may be bonded to the first holders 132, 133. In addition, the elastic supports 143, 144 may be bonded to the photocatalyst carrier 131. In the configuration of the elastic supports 141 to 144, all the elastic supports 141 to 144 may be bonded to the photocatalyst carrier 131. Alternatively, all the elastic supports 141 to 144 may be bonded to the first holders 132, 133 and the second holders 134, 135 without bonded to the photocatalyst carrier 131. The elastic supports 141 to 144 are only required to at least partially contact an outer peripheral portion of the photocatalyst carrier 131. That is, the elastic supports 141 to 144 do not necessarily contact the entire circumference of the outer peripheral portion.

In the above-described embodiment, the elastic supports 141 to 144 are arranged between the photocatalyst carrier 131 and the first holders 132, 133 and between the photocatalyst carrier 131 and the second holders 134, 135. However, the elastic supports 141 to 144 are only required to elastically support the photocatalyst carrier 131. Thus, the elastic supports 141 to 144 do not necessarily contact the photocatalyst carrier 131. For example, the elastic supports 141 to 144 can also be bonded to an outer peripheral portion of the photocatalytic unit 130 as in the elastic supports 145, 146. In this manner, the elastic supports 141 to 144 can also be arranged between the housing 102 and the photocatalytic unit 130.

In the above-described embodiment, the elastic supports 141 to 144 are made of an open-cell foam resin material. With this configuration, the elastic supports 141 to 144 can effectively vibrate the photocatalyst carrier 131. However, the elastic supports 141 to 144 may be made of a closed-cell foam resin material. Alternatively, the elastic supports 141 to 144 can also be formed of elastic bodies with no cells. Alternatively, the elastic supports 141 to 144 can also be formed of coil springs or plate springs.

In the above-described embodiment, in the air purifier 100, the light source boards 120 are arranged so as to face both surfaces of the photocatalytic unit 130. With this configuration, both surfaces of the photocatalytic unit 130 are irradiated with light. However, in the configuration of the air purifier 100, only one of both surfaces of the photocatalytic unit 130 may be irradiated with light.

In the configuration of the air purifier 100, the light source boards 120 are arranged so as to face both surfaces of the photocatalytic unit 130. With this configuration, both surfaces of the photocatalytic unit 130 are irradiated with light. As a result, the suction port 103 and the discharge port 106 are also irradiated with light. Thus, light leaks to the outside of the air purifier 100 through the suction port 103 and the discharge port 106. Thus, the actuation status of the air purifier 100 can be easily checked. However, the air purifier 100 can also be configured such that no light leaks to the outside through the suction port 103 and the discharge port 106. The light source 120a is only required to emit light for exerting the photocatalytic action to the photocatalyst. Thus, the air purifier 100 may be configured such that the photocatalytic unit 130 is irradiated with light (e.g., ultraviolet light) other than visible light according to the photocatalyst, needless to say.

In the above-described embodiment, the air path forming member 110 includes the windward plates 112, 116. With this configuration, the sectional area of the flow path R decreases. In addition, the air path forming member 110 includes the leeward plates 114, 118. With this configuration, the sectional area of the flow path R increases. However, the air path forming member 110 can also be formed with the flow path R having a constant sectional area between the suction port 103 and the discharge port 106.

The foregoing detailed description has been presented for the purposes of illustration and description. Many modifications and variations are possible in light of the above teaching. It is not intended to be exhaustive or to limit the subject matter described herein to the precise form disclosed. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims appended hereto.

What is claimed is:

1. A photocatalytic air purifier comprising:
in a housing, a photocatalyst that purifies air and a light source that irradiates the photocatalyst with light exerting a photocatalytic action,
wherein the photocatalyst is elastically supported in the housing.

2. The photocatalytic air purifier according to claim 1, further comprising:
an air blower that sends air to a photocatalyst side or sucks air from the photocatalyst side.

3. The photocatalytic air purifier according to claim 1, further comprising:
a photocatalytic unit,
wherein the photocatalytic unit is attached in the housing, and supports the photocatalyst, and
the photocatalyst is elastically supported in the photocatalytic unit.

4. The photocatalytic air purifier according to claim 3, wherein
the photocatalytic unit is attachable to and detachable from the housing.

5. The photocatalytic air purifier according to claim 1, wherein
the photocatalyst is directly or indirectly supported, at at least part of an outer peripheral portion of the photocatalyst, by the housing through elastomer or rubber.

6. An air purification method comprising:
using a photocatalytic air purifier including, in a housing, a photocatalyst that purifies air and a light source that irradiates the photocatalyst with light exerting a photocatalytic action;
elastically supporting the photocatalyst in the housing; and
vibrating the photocatalyst.
